(19) United States
(12) Patent Application Publication
(10) Pub. No.: US 2010/0168411 A1
(43) Pub. Date: Jul. 1, 2010

(54) Method of producing fermentation product and fermentation product

(75) Inventor: Kokichi Hanaoka, Nagano (JP)

Correspondence Address: LEE FREDRIC SHARRA, 4313 TROUTHAVEN DRIVE, MURRYSVILLE, PA, 15668, US

(73) Assignees: Kokichi HANAOKA, Nagano (JP); Yoshiaki MATSUO, Tokyo (JP); Ryouichi OHTSUBO, Tokyo (JP); Atsuyoshi MURAKAMI, Tokyo (JP); Yuu KOYAMA, Tokyo (JP)

(21) Appl. No.: 12/663,002

(22) PCT Filed: Apr. 15, 2009
(86) PCT No.: PCT/JP2009/001743

§ 371 Date: Dec. 4, 2009

(30) Foreign Application Priority Data
Apr. 15, 2008 (JP) 2008-105458

(51) Int. Cl. C08B 37/00; C12P 1/00

(52) U.S. Cl. 536/123.1; 435/41

(57) ABSTRACT

Provided are a fermentation product, which shows a strong antioxidative effect and is efficacious against diseases caused by active oxygen, and a method of producing the same. Stems and leaves of a perennial gramineous plant (rice straw, reed, barely straw, etc.) are mixed with a fermentation medium which comprises egg albumen, egg yolk, rice bran and water optionally together with xylase and thus the stems and leaves of the perennial gramineous plant are decomposed. After conducting lactic acid fermentation, the contents of the fermentation tank are dried. The dry fermentation product thus obtained is extracted with water and the extract is filtered and then concentrated.

### DETAILED DESCRIPTION OF THE INVENTION

1. Field of the Invention

The present invention relates to a production method and a fermentation product, in particular, using a perennial plant gramineae fiber as a starting material and using a microbe for lactic acid fermenting.

2. Background Art

The inventor of this present application proposed a production method of a water-soluble polysaccharide which is effective for the treatment of the Hepatitis B and the water-soluble polysaccharide in patent document 1 and patent document 2 previously. Patent document 1 and patent document 2 disclose the following: A gramineae vegetable fiber is immersed in an ammonia-related nitrogen, a nitric acid-related nitrogen, a soluble phosphoric acid, and a culture fluid including a soluble potassium. Fermentation bacteria attached to the gramineous plant make the gramineous fiber ferment. When a pH level of the culture fluid reaches from 7-9.2, fermentation is stopped. As a result, the molecular weight of the main ingredient becomes 70×10³ or more by conversion of dextran. And polysaccharide containing a secondary amine ingredient is obtained.
[Patent Document 1] JPA 59-143598
[Patent Document 2] JPA 05-310802

### DISCLOSURE OF THE INVENTION

### Problem to be Solved by the Invention

In the above mentioned methods described in patent document 1 and patent document 2, a culture fluid is required to be prepared beforehand. However, the preparation of the culture fluid takes effort. Also, since there is only a little yield (fermentation product/whole weight) of the fermentation product, and because the fermentation is performed using a prepared culture fluid, the result is the problem that a profit can not be produced.

### Means to Solving the Problem

The production method of the fermentation product of the present invention has the following features to solve the problem. For the purpose of a starting material, the leaf stem of a perennial gramineae grass plant, for example, rice straw, a reed straw, was used. As a fermentation nutrient medium, an egg white, an egg yolk, rice bran and water were used, alternatively xylase may be added. The fermentation nutrient medium and the leaf stem of the perennial gramineae plant were mixed. After the leaf stem of the rice straw is broken down, and lactic acid ferment acid is made, a dry fermentation product is obtained by drying a product in the fermenter. The dried fermentation product is then extracted by means of water extraction. The extract is then condensed through filtration.

In addition, the fermentation product provided in this manufacturing method, the main ingredient is converted into dextran made up of a polysaccharide having the molecular weight approximately 70×10³ and a polysaccharide having the molecular weight of approximately 500×10³, with glucose and fructose as monosaccharides.

### Effect of the Invention

In accordance with this invention, the provided fermentation product (polysaccharide+monosaccharides) is effective in the treatment of illnesses derived from active oxygen, in addition to its curative effect for hepatitis B as inspected with patent document 1 or 2, but also a more acid-fast voltinism (neutralizing active oxygen) is recognized.

A secondary amine is contained in the fermentation product disclosed in patent documents 1 and 2, but in the present invention the secondary amine is not contained in the fermentation product. With regard to the present invention, the chromatographic wave pattern is relatively broad. Note that it is unknown whether a fermentation product disclosed in patent document 1 and 2 had an acid-fast voltinism.

That is, the fermentation product of the present invention generates makes a super oxide radical which is imbalanced. Additionally the super oxide radical is eliminated, and the toxic strong hydroxy radical can be eliminated. Therefore, it is an extremely efficient antioxidant. Besides, because it is a fermentation product derived from a natural product, it is safe, and there is no toxicity. The super oxide radical is a kind of active oxygen generated by 1 electron reduction of oxygen molecules, and the hydroxy radical is a kind of active oxygen generated by a 3 electron reduction of oxygen molecules.

Particularly, the amino acids, lipids and minerals which are necessary for natural fermentation are included abundantly in the egg white, egg yolk and rice bran which is the fermentation nutrient medium. However, since the substrate of the leaf stem of the perennial gramineae plant is comprised of lignin and hemicellulose including xylose fermentation completion can require a long time, but fermentation resolution efficiency can be raised by adding xylase which is a xylose degrading enzyme. Furthermore, the fermentation is eco-friendly, and energy cost can be inexpensive because the process can be conducted with natural sun light.

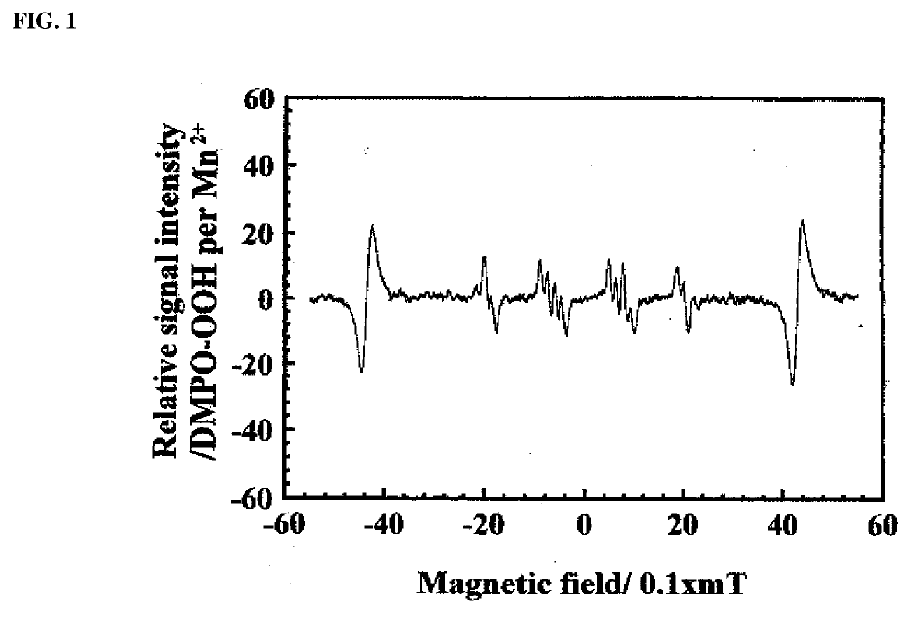

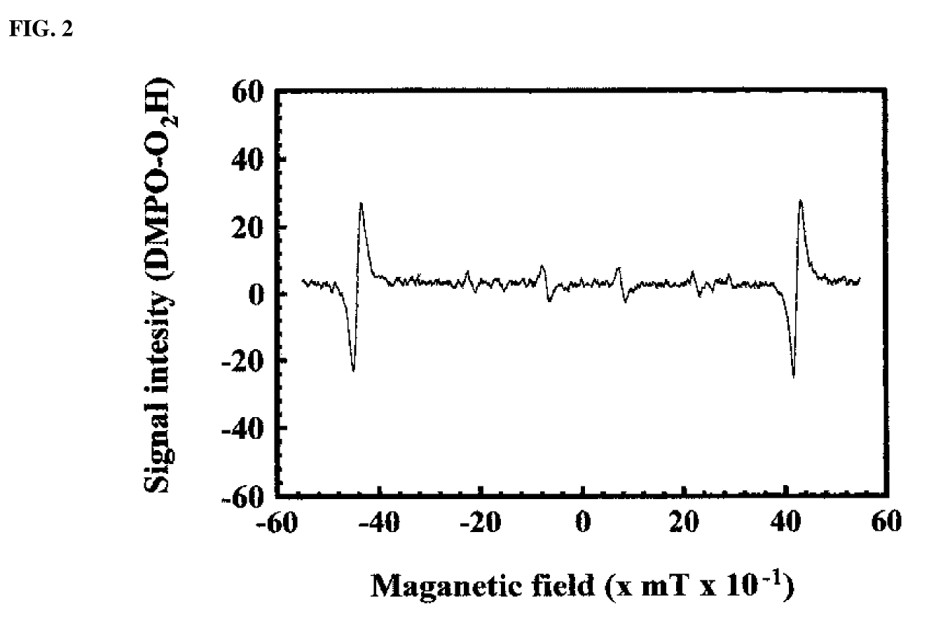

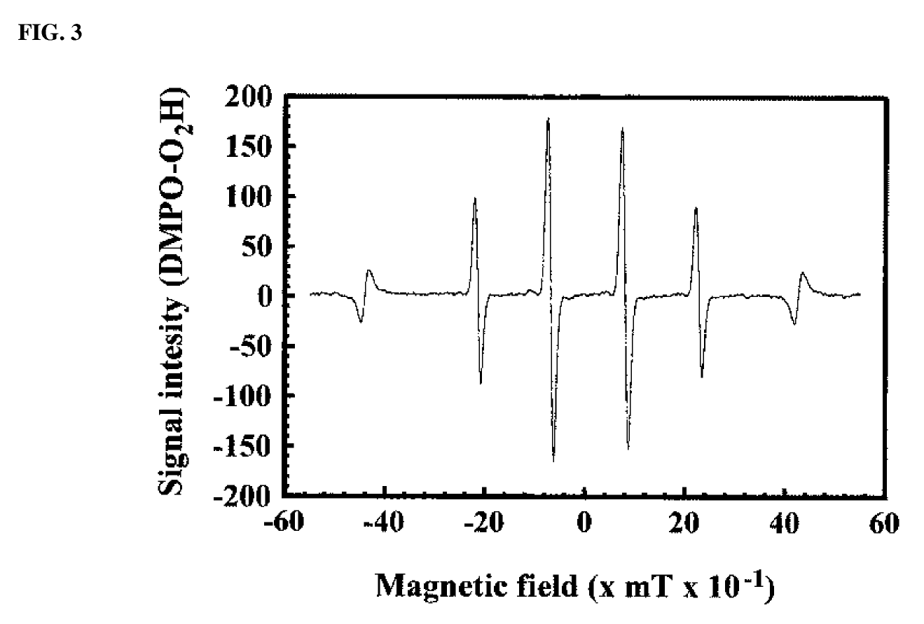

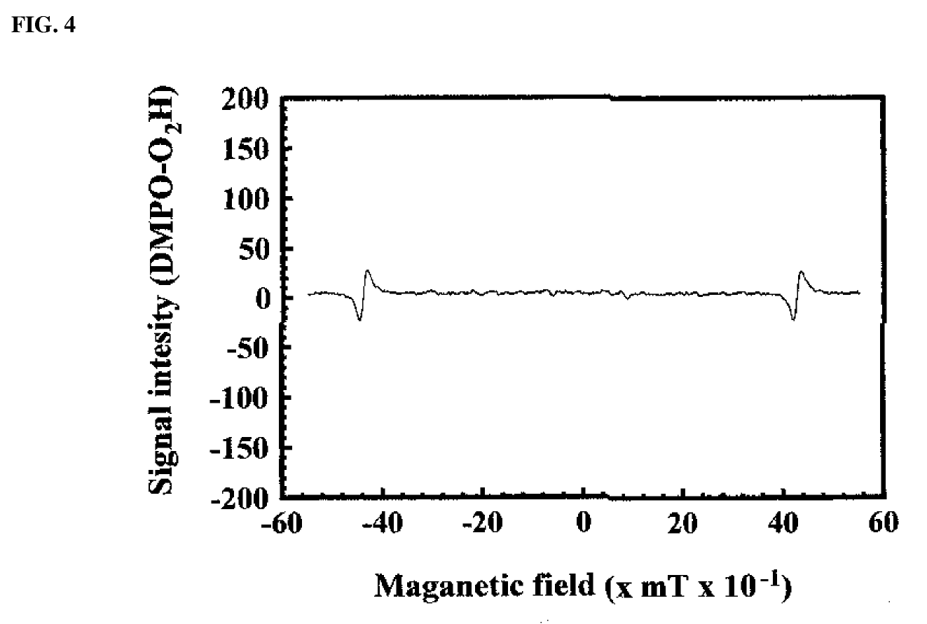

### BRIEF DESCRIPTION OF DRAWINGS

[FIG. 1] A graph showing the strength of the ESR (Electron Spin Resonance) of a super oxide radical formed by hypoxanthine-xanthine oxidase.

[FIG. 2] A graph showing the strength of the ESR of a super oxide radical erased by a fermentation product concerning the present invention.

[FIG. 3] A graph showing the strength of the ESR of a hydroxy radical generated by hydrogen peroxide and ferrous sulfate.

[FIG. 4] A graph showing the strength of the ESR of a hydroxy radical erased by a fermentation product concerning the present invention

### BEST MODE FOR CARRYING OUT THE INVENTION

### EXAMPLE 1

40 L of water was introduced into a culture tank of 60 cm in width×100 cm in length×20 cm in height. 200 g of rice bran and 4 chicken eggs (egg white and egg yolk) stirred well were added, and it was mixed, and the culture fluid was adjusted.

Rice straw was cut into 3 cm lengths so that it might be able to be mixed well with the culture fluid and it was mixed with culture fluid.

Using the cover so that light was possible to enter, light of the sun was guessed right thoroughly, and it made lactic fermentation takes place. Then it was stirred every 2nd day, and fermentation was promoted.

The fermentation bacteria to perform lactic fermentation are Lactobacillus, Bifidobacterium, Lactococcus, Pediococcus, Leuconostoc. It is thought that the one or more of these bacteria attaches to rice straw.

When culture fluid shifts to the alkali domain by lactic fermentation from the acidity domain, and a color changed to strong tea dark brown color, it is gradually evaporated, and it is assumed a dry state.

The dried fermentation product is water soluble and is water extracted. The solution containing the extracted water-soluble ingredient is then filtered. The filtrate is heat concentrated, and the water becomes around 70% concentrated. The filtrate is further concentrated to 50%-60% of water in a dryer of temperature 130 degrees Celsius, and it assumes the form of a paste.

Then the paste-like fermentation product is solidified with 95% ethyl alcohol. The fermentation product is water-soluble, but it is insoluble in alcohol.

Furthermore, after having solidified with ethyl alcohol, it is filtered, and a solid filtration product is obtained. After this solid filtration product was dried at 80 degrees Celsius, and having evaporated the ethyl alcohol completely, a solid fermentation product containing 1% or less of water is obtained.

Weight of the whole solid of rice straw and the nutrient medium was 1,940 g, and the yield of the provided solid fermentation product was 380 g on the average. In the case of a method of patent documents 1 and 2, the yield of the provided solid fermentation product was an average of 170 g. The yield of a provided solid fermentation product improves by greater than double.

The solid fermentation product that manufactured by the above-mentioned method was dissolved in pure water, and a sample of the 0.1% density was obtained. The pure water was processed a reverse osmotic membrane and deionization. And the super oxide radical which was formed by hypoxanthine-xanthine oxidase system, the elimination ability of a hydroxy radical formed by Fenton reaction was examined by Electron Spin Resonance method (ESR).

FIG. 1 is a signal showing the strength of the ESR of a super oxide radical formed by hypoxanthine xanthine oxidase, and FIG. 2 is a signal of ESR eliminated by the sample. The fermentation product provided by the present invention as shown by figures one and two show strong elimination ability of free radicals.

Additionally, FIG. 3 shows a signal depicting the strength of a hydroxy radical generated by hydrogen peroxide and ferrous sulfate, and FIG. 4 is of a signal depicting the strength to which a hydroxy radical was eliminated by the sample. The fermentation product provided by the present invention showed strong elimination ability of a hydroxy radical, as shown by figures three and four.

The molecular weight of a fermentation product provided with Example 1 was measured in the following condition using high-speed liquid chromatography (a Hitachi 635 type).
Colum Shodex Ionpak: s′800p+s′804+s′801
Sample: 10 μl
Detector: RI4k×10-5 RLUFS′
Pressure: 25 kg/cm2
Eluent: H2O
Flow Rate: 1.0 ml/min
Chart speed: 5 mm/min
Colom temp: 60° CRT

The results of measurement using high speed liquid chromatography show that the overall shape becomes broader than a shape of the sample as disclosed in patent document 1 and 2. However, it is similar and at the position localized for where a peak of dextran of the molecular weight 70×10³ and a peak for dextran of the molecular weight 500×10³ would be found two principal peaks emerge. Furthermore, glucose and fructose were detected as two monosaccharides, as well.

The product which dissolved 3 mg of provided solid fermentation product in 100 mL of pure water was assumed a sample. A SOD Assay Kit-WST made by Dojin Laboratories Co., Ltd., was used and the absorbance measurement of the wavelength was 450 nm. This was performed using a microplate reader made by Corona Electric Corporation Co., Ltd. As a result, the check rate of the super oxide radical was 20.6%.

### EXAMPLE 2

Water 40 L was introduced into a culture tank of 60 cm in width×100 cm in length×20 cm in height. 200 g of rice bran and four chicken eggs and 15 g xylase and this was mixed preferably by stirring, and culture fluid was adjusted.

Rice straw was prepared by cutting into 3 cm lengths so that it might be able to be mixed well with the culture fluid and it was mixed with culture fluid.

Using the cover so that light was possible to enter, light of the sun was guessed right thoroughly, and it made lactic fermentation takes place. Then it was stirred every 2nd day, and fermentation was promoted.

In a procedure like Example 1, a solid fermentation product 1% of water or less was obtained. The yield was 450 g on the average, and it was recognized that a yield further improved by adding xylase.

The fermentation product showed the strong ability to elimination free radicals when the elimination ability of the super oxide radical and the hydroxy radical was examined by the electron spin resonance method (ESR) on this solid fermentation product in a procedure like Example 1.

Also, in a procedure like Example 1, a check rate of the super oxide radical was verified, and the result was 25.4%. As compared with Example 1, it is found that 23.3% SOD of activity improves in comparison with an additive-free fermentation article by adding xylase which is one of the fiber degrading enzymes.

What is claimed is:

1. A method of producing a fermentation product having antioxidant functionality comprising:
Mixing rice straw leaf stem and a fermentation nutrient medium comprising egg white, egg yolk, rice bran and water in a fermenter,
Permitting time for the rice straw leaf stem to be broken down, and lactic acid to ferment, then obtaining a dry fermentation product through in fermenter drying,
Extracting a dry fermentation product by water extraction, and,
Condensing said dry fermentation product through filtration.

2. A method of producing a fermentation product extract having antioxidant functionality comprising:
Mixing rice straw leaf stem and a fermentation nutrient medium: comprising egg white, egg yolk, rice bran, xylan and water;
Permitting time for the rice straw leaf stem to break down and for lactic acid to ferment, and drying a resultant product in the fermenter to obtaining a dry fermentation product;
Extracting the dry fermentation product by water extraction, and
Condensing the fermentation product extract through filtration condensation.

3. A fermentation product generated by lactic fermentation of a mixture of rice straw leaf stem and a fermentation nutrient medium that is comprised of egg white, egg yolk, rice bran and water comprising the following characteristics:
Where the molecular weight of the chief ingredient converts into dextran having a polysaccharide of 70×10³ and a polysaccharide 500×10³, and contains glucose and fructose. A fermentation product generated by the lactic fermentation of a mixture of rice straw leaf stem and a fermentation nutrient medium comprised of egg white, egg yolk, rice bran, xylase and water comprising following characteristics:
Wherein the molecular weight of the chief ingredient converts to dextran, it is polysaccharide of 70×10³, and a polysaccharide of 500×10³, and contains glucose and fructose.
